(19) United States
(12) Patent Application Publication
(10) Pub. No.: US 2024/0058428 A1
(43) Pub. Date: Feb. 22, 2024

(54) COMPOSITION FOR TREATING OR PREVENTING MYOPATHY, OBESITY OR DIABETES

(71) Applicant: NUON BIO CO.,LTD., Gyeonggi-do (KR)

(72) Inventors: Bong Keun CHOI, Gyeonggi-do (KR); Sung Kwon LEE, Gyeonggi-do (KR); Sang Hyup LEE, Gyeonggi-do (KR)

(21) Appl. No.: 18/267,252

(22) PCT Filed: Dec. 14, 2021
(86) PCT No.: PCT/KR2021/018986

§ 371 Date: Jun. 14, 2023

(30) Foreign Application Priority Data
Dec. 14, 2020 (KR) 10-2020-0174841

(51) Int. Cl. A61K 38/48; A61P 21/00; C12N 9/64

(52) U.S. Cl. CPC A61K 38/4886; A61P 21/00; C12N 9/6489

(57) ABSTRACT

The present invention relates to: an M3 mutant peptide; a composition for alleviating, inhibiting, preventing or treating myopathy, obesity or diabetes, comprising same as an active ingredient; a method for alleviating, inhibiting, preventing or treating myopathy, obesity or diabetes by using the M3 mutant peptide; and uses of the M3 mutant peptide for alleviating, inhibiting, preventing or treating myopathy, obesity or diabetes.

### TECHNICAL FIELD

This application claims priority and the benefit of Korean Patent Application No. 10-2020-0174841 filed in the Korean Intellectual Property Office on 14 Dec. 2020, the disclosure of which is incorporated herein by reference.

The present disclosure relates to an M3 mutant peptide, a composition comprising the same as an active ingredient for alleviation, suppression, prevention, or treatment of a muscle disease, obesity, or diabetes, a method for alleviating, suppressing, preventing, or treating a muscle disease, obesity, or diabetes by using an M3 mutant peptide, and use of an M3 mutant peptide for alleviating, suppressing, preventing, or treating a muscle disease, obesity, or diabetes.

### BACKGROUND

A reduction in human physical activity is a result of lifestyle changes due to the industrialization in modern society, and it has been a long time since the issue of negative effects on physical and mental health has been raised. Lack of exercise caused by reduced physical activity is a major cause of various lifestyle diseases including obesity and metabolic syndrome and causes a decrease in muscle mass. The muscle mass in the body plays a very important role in survival, and a decrease in skeletal muscle volume leads to a decrease in basal metabolic rate, a decrease in muscle strength, and the like, thereby making it difficult to maintain daily physical activity.

Muscle atrophy refers to a decrease in muscle volume, and symptoms thereof can be clearly evidently seen in patients suffering from cancer or various immunodeficiency diseases and are more prominent in patients with extremely restricted physical activity due to long-term bed life, physical handicapped person with restricted movement, and the elderly. The progression of aging causes sarcopenia indicating a decrease in muscle strength due to a continuous decrease in muscle volume, causing dangerous results that the elderly may be exposed to various life accidents.

With advancing years, both men and women undergo a change in body composition, and such a change occurs independently of the body weight change. In general, the change in body composition is considered to be due to a change in energy balance due to a weight gain, and the change in body composition appearing herein results in an increase in body fat but a decrease in muscle mass. The decrease in muscle mass gradually progresses from about 30 years of age, and sarcopenia in the elderly seriously affects functional disability, quality of life, and medical costs.

An increase in fat mass together with a decrease in muscle mass is referred to as sarcopenic obesity. A synergistic effect of an increase in body fat and a muscle atrophy will further increase the risk of functional disorders and metabolic disorders in the body.

Body mass index (BMI) and waist to hip ratio (WHR), which are used as indicators of obesity due to ease of access thereto in clinical practice, have limitations in that they do not reflect well the effects of age or exercise, changes in body composition due to weight loss, and the like. In particular, the muscle mass in the body composition has been reported to be related to metabolic syndrome and is suggested as a factor predicting the cause of all mortality. In recent years, in terms of health, there is an increasing need to pay attention to an increase in body fat percentage and a loss of muscle mass as factors related to insulin resistance, glucose metabolism, lipid concentration, and blood pressure.

Throughout the entire specification, many papers and patent documents are referenced and their citations are represented. The disclosures of cited papers and patent documents are entirely incorporated by reference into the present specification, and the level of the technical field within which the present disclosure falls and details of the present disclosure are explained more clearly.

### DISCLOSURE OF INVENTION

### Technical Problem

Therefore, in order to develop an agent for alleviation, suppression, prevention, or treatment of a muscle disease related to muscle loss, the present inventors screened for peptides having possibility of showing an effect against muscle loss, found that among the screened peptides, an M3 mutant peptide had significant muscle loss inhibitory activity, isolated and identified proteins having activity, and confirmed that the identified mutant peptides had superior activity compared with currently used medicines for muscle diseases related to muscle loss, and thereafter completed the present disclosure.

Accordingly, an aspect of the present disclosure is to provide an M3 mutant peptide.

Another aspect of the present disclosure is to provide a pharmaceutical composition comprising an M3 mutant peptide as an active ingredient for alleviation, suppression, prevention, or treatment of a muscle disease.

Still another aspect of the present disclosure is to provide a method for alleviating, suppressing, preventing, or treating muscle loss, the method including administering to a subject in need thereof an M3 mutant peptide in an amount effective to alleviate, suppress, prevent, or treat a muscle disease.

Still another aspect of the present disclosure is to provide use of an M3 mutant peptide for alleviation, suppression, prevention, or treatment of a muscle disease.

Aspect of the present disclosure is to provide a pharmaceutical composition comprising an M3 mutant peptide as an active ingredient for alleviation, suppression, prevention, or treatment of obesity.

Another aspect of the present disclosure is to provide a method for alleviating, suppressing, preventing, or treating obesity, the method including administering to a subject in need thereof an M3 mutant peptide in an amount effective to alleviate, suppress, prevent, or treat obesity.

Still another aspect of the present disclosure is to provide use of an M3 mutant peptide for alleviation, suppression, prevention, or treatment of obesity

Aspect of the present disclosure is to provide a pharmaceutical composition comprising an M3 mutant peptide as an active ingredient for alleviation, suppression, prevention, or treatment of diabetes.

Another aspect of the present disclosure is to provide a method for alleviating, suppressing, preventing, or treating diabetes, the method including administering to a subject in need thereof an M3 mutant peptide in an amount effective to alleviate, suppress, prevent, or treat diabetes.

Still another aspect of the present disclosure is to provide use of an M3 mutant peptide for alleviation, suppression, prevention, or treatment of diabetes.

Other purposes and advantages of the present disclosure will become more obvious when taken with the following detailed description of the invention, claims, and drawings.

### Solution to Problem

The present disclosure relates to an M3 mutant peptide, a composition comprising the same as an active ingredient for alleviation, suppression, prevention, or treatment of a muscle disease, obesity, or diabetes, and a method for alleviating, suppressing, preventing, or treating a muscle disease, obesity, or diabetes, by using an M3 mutant peptide.

Hereinafter, the present disclosure will be described in more detail.

In accordance with an aspect of the present disclosure, there is provided an M3 mutant peptide.

The M3 mutant peptide of the present disclosure is a mutant peptide of a wild-type of a disintegrin and metalloproteinase with thrombospondin motifs 1 (ADAMTS1).

ADAMTS1 is an enzyme that belongs to a disintegrin and metalloproteinase with thrombospondin motifs (ADAMTS), a family of multidomain extracellular protease, and is first found among 19 species of ADAMTS family enzymes discovered in humans. ADAMTS1 is known to inhibit angiogenesis by interaction with vascular endothelial growth factor A.

In the present disclosure, the M3 mutant peptide may be a peptide in which a portion of a peptide consisting of the amino acid sequence of SEQ ID NO: 1 is deleted.

In one specific embodiment of the present disclosure, the M3 mutant peptide of the present disclosure may include a deletion of at least one amino acid selected from the group consisting of amino acids at positions 223 to 261 of the peptide consisting of the amino acid sequence of SEQ ID NO: 1.

In one specific embodiment of the present disclosure, the M3 mutant peptide of the present disclosure may include a deletion of at least one position selected from the group consisting of arginine at position 249, lysine at position 250, lysine at position 251, and arginine at position 252 of the peptide consisting of the amino acid sequence of SEQ ID NO: 1, and for example, may include deletions of positions 249 to 251.

In one specific embodiment of the present disclosure, the M3 mutant peptide of the present disclosure may include a deletion of at least one position selected from the group consisting of arginine at position 249, lysine at position 250, lysine at position 251, and arginine at position 252 and a deletion of at least one amino acid selected from the group consisting of amino acids at positions 223 to 248 and 253 to 261 of the peptide consisting of the amino acid sequence of SEQ ID NO: 1. For example, the M3 mutant peptide of the present disclosure may include a deletion of at least one position selected from the group consisting of arginine at position 249, lysine at position 250, lysine at position 251, and arginine at position 252 and a deletion of at least one amino acid selected from the group consisting of amino acids at positions 223 to 248 of the peptide consisting of the amino acid sequence of SEQ ID NO: 1.

In the present disclosure, the deletion of at least one amino acid selected from the group consisting of the amino acids at positions 223 to 248 may be a deletion of an amino acid at position 223, or deletions of amino acids at positions 223 to 224, 223 to 225, 223 to 226, 223 to 227, 223 to 228, 223 to 229, 223 to 230, 223 to 231, 223 to 232, 223 to 233, 223 to 234, 223 to 235, 223 to 236, 223 to 237, 223 to 238, 223 to 239, 223 to 240, 223 to 241, 223 to 242, 223 to 243, 223 to 244, 223 to 245, 223 to 246, 223 to 247, or 223 to 248.

In the present disclosure, the deletion of at least one amino acid selected from the group consisting of amino acids at positions 253 to 261 may be a deletion of amino acid at position 253, or deletions of amino acids at positions 253 to 254, 253 to 255, 253 to 256, 253 to 257, 253 to 258, 253 to 259, 253 to 260, or 253 to 261.

In one specific embodiment of the present disclosure, the M3 mutant peptide of the present disclosure may further include deletions of aspartic acid at position 413 and serine at position 967 of the peptide consisting of the amino acid sequence of SEQ ID NO: 1.

In one specific embodiment of the present disclosure, the M3 mutant peptide of the present disclosure may include deletions of positions 223 to 251 and deletions of positions 413 to 967 of the peptide consisting of the amino acid sequence of SEQ ID NO: 1, and for example, may be a peptide consisting of the amino acid sequence of SEQ ID NO: 2.

In accordance with another aspect of the present disclosure, there is provided a pharmaceutical composition comprising the M3 mutant peptide of the present disclosure as an active ingredient for alleviation, suppression, prevention, or treatment of a muscle disease.

In the present disclosure, the M3 mutant peptide is as described above.

The distribution of body mass in muscle and adipose tissues has a significant influence on individual health. The balance between adipocyte differentiation and myocyte differentiation is known to involve many factors. For example, glucocorticoids, such as cortisol, natural hormones, or many synthetic cortisol analogues (including, e.g., prednisone, hydrocortisone, and dexamethasone), act on the body through the glucocorticoid receptor (GR). Glucocorticoids play an important role in regulating differentiation determination both in vivo and ex vivo, and promote adipogenesis and inhibit myogenesis. Androgen administration has been shown to influence the body composition to increase muscle mass while reducing fat mass.

Muscle insufficiency or weakness is one of the most devastating childhood health problems. These muscle disorders affect more than 1 in 3000 patients with various congenital myopathy and muscular dystrophy, such as Duchenne Muscular Dystrophy (DMD). These disorders are typically associated with genetic or spontaneous gene mutations. Children with these disorders suffer from a wide range of complications. Currently, the lack of effective therapy results in a high mortality rate, and new treatment strategies for such muscle diseases are needed.

The muscle tissue of adult vertebrates is regenerated from stem cells known as satellite cells or muscle stem cells (MuSCs). Satellite cells are distributed throughout muscle tissue, mitotically quiescent in the absence of injury or disease, and located in anatomically defined niches. In addition to the satellite cells, the type of cells that can contribute to muscle regeneration includes mesenchymal stem cells, bone marrow derived cells, muscle stromal cells, and mesenchymal stem cells, but are not limited thereto.

Tissue engineering involves the restoration or replacement of damaged or diseased tissues by a transplantation of a combination of cells, biomaterial scaffolds, biological active molecules, and genes. The basic premise of this approach is that exogenously introduced cells will improve the rate and extent of tissue repair.

Adult MuSCs can be transplanted into damaged or defective skeletal muscles to reconstruct muscle fibers and enhance functions, thereby potentially providing therapeutic applications of MuSCs. However, major obstacles in translating this technology are that there is a lack of understanding of how differentiation determinations are made, and no tools for controlling and promoting these determinations for therapeutic advantages have not been developed. Advances in these fields can explore a variety of new therapies for individuals with impaired muscle mass or functions or resolve the imbalance between muscle and adipose tissues.

In the present disclosure, the muscle disease may be selected from the group consisting of sarcopenia, amyotrophy, cancer cachexia, muscle injury, muscular dystrophy, atrophy of heart, atony, muscular dystrophy, muscular degeneration, and myasthenia, but is not limited thereto, and includes all of muscle diseases related to muscle loss.

Sarcopenia was first named by Rosenberg in 1989, and the term sarcopenia is a compound word of “sarco” meaning muscle and “penia” meaning a reduction, these being originated from Greek. In early 2017, the World Health Organization (WHO) assigned a disease classification code to sarcopenia and officially recognized it as a condition characterized by lower muscle mass than normal. Sarcopenia refers to the loss of skeletal muscle mass mainly distributed in the limbs and results from gradual loss of skeletal muscle mass associated with aging.

Amyotrophy is defined as a reduction in muscle mass. Amyotrophy is often developed when a patient is at rest, such as being in the hospital, or when a patient is restrictively moving That is, amyotrophy is often developed in cachexia where a patient is inactive or suffers from two or more diseases, such as cancer, AIDS, congestive heart failure, chronic obstructive pulmonary disease, renal failure, and severe burns. Amyotrophy is known to increase the risk of a fall and a hurt from a fall and decrease energy consumption, which is resultantly associated with increasing the risk of obesity and metabolic diseases. The results of severe diseases, such as cancer, which is a metabolic disease, may also cause amyotrophy, and this was proved to be induced by an increase in inflammatory cytokines and activation of inflammatory signaling pathways. Similarly, an environment of obesity also increases the generation of reactive oxygen species inducing oxidative stress, induces metabolic changes, such as increased influx of fatty acids into muscle tissue and reduced fatty acid oxidation, and causes disorders in mitochondrial biosynthesis, structure, and function. These oxidative stress and influx of fatty acids in the muscle may also cause inflammation and cause amyotrophy.

In accordance with another aspect of the present disclosure, there is provided a method for alleviating, suppressing, or treating a muscle disease, the method including administering to a subject a pharmaceutical composition comprising an M3 mutant peptide as an active ingredient.

In accordance with still another aspect of the present disclosure, there is provided a composition comprising an M3 mutant peptide as an active ingredient for alleviation, suppression, prevention, or treatment of obesity.

In the present disclosure, the M3 mutant peptide is as described above.

As used herein, the term “obesity” refers to a condition in which adipose tissues are excessively accumulated in the body so as to cause health disorders.

In the development of obesity, an increase in adipose tissue mass may be due to increase in the size and number of adipocytes. The increase in cell number may result in the mobilization of pre-adipose cells from a pluripotent stem cell population or from a subpopulation of cells residing in mature white adipose tissue (WAT). Bone marrow-derived mesenchymal stem cells (BM-MSCs) may differentiate into various cell types including fat, muscle, cartilage and bone. With aging, bone marrow adipogenesis accelerates in vivo, while the bone formation ability of MSCs decreases. It has been suggested that MSC precursors may contribute to age-related body composition changes by differentiating into adipose rather than bone, which is in a reciprocal relationship state.

Fat redistribution in the elderly is associated with an increased risk of metabolic syndrome, including diabetes, hypertension, dyslipidemia, atherosclerosis, and relatively increased intra-abdominal fat. In addition, muscle aging and normal aging-related muscle performance decline occur, often with a gradual onset of sarcopenia. Skeletal muscles have self-renewal ability, but this process is not activated in the elderly. Age-related changes within skeletal muscle tissues and host environments are known to influence myoblast proliferation and fusion in response to injury in aged animals.

In accordance with another aspect of the present disclosure, there is provided a method for alleviating, suppressing, or treating obesity, the method including administering to a subject a pharmaceutical composition comprising an M3 mutant peptide as an active ingredient.

In accordance with still another aspect of the present disclosure, there is provided a composition comprising an M3 mutant peptide as an active ingredient for alleviation, suppression, prevention, or treatment of diabetes.

In the present disclosure, the M3 mutant peptide is as described above.

As used herein, the term “diabetes” refers to a chronic disease characterized by a relative or absolute shortage in insulin, causing glucose-intolerance. The term diabetes includes all types of diabetes, for example, Type 1 diabetes, Type 2 diabetes, or hereditary diabetes. Type 1 diabetes is the insulin-dependent diabetes, which is mainly caused by β-cell disruption. Type 2 diabetes is the insulin-independent diabetes, which is caused by an insufficient secretion of insulin after eating or by insulin tolerance.

In accordance with still another aspect of the present disclosure, there is provided a method for alleviating, suppressing, or treating diabetes, the method including administering a pharmaceutical composition comprising an M3 mutant peptide as an active ingredient.

As used herein, the term “comprising as an active ingredient” refers to the inclusion of an amount that is sufficient to attain the efficacy or activity of the M3 mutant peptide.

The content of the peptide as an active ingredient in the composition of the present disclosure can be appropriately adjusted depending on the type and purpose of use, patient condition, type and severity of symptom, and the like. The content may be, based on the weight of solids, 0.001 to 99.9 wt %, 0.1 to 99.9 wt %, 1 to 90.9 wt %, 0.001 to 99 wt %, 0.1 to 99 wt %, 1 to 99 wt %, 0.001 to 90 wt %, 0.1 to 90 wt %, 1 to 90 wt %, 0.001 to 80 wt %, 0.1 to 80 wt %, 1 to 80 wt %, 0.001 to 70 wt %, or 0.1 to 70 wt %, for example, 1 to 70 wt %, but is not limited thereto, and may be selected within an appropriate range by a person skilled in the art.

The composition according to the present disclosure may be administered to mammals including humans through various routes. The manner of administration may be any manner that is conventionally used. For example, the administration may be performed through an oral, dermal, intravenous, intramuscular, or subcutaneous route, and preferably, an intravenous route.

The composition of the present disclosure may be formulated in the form of: an oral formulation, such as a powder, granules, a tablet, a capsule, a suspension, an emulsion, a syrup, or an aerosol; a parenteral formulation, such as a transdermal preparation, an ointment, a suppository, or a sterile injectable solution, according to a conventional method for each case.

The composition of the present disclosure may further comprise an adjuvant, such as a pharmaceutically suitable and physiologically acceptable carrier, excipient, and diluent, in addition to the mixed extract.

Examples of the carrier, excipient, and diluent that may be contained in the composition of the present disclosure may include dextrin, crystalline cellulose, lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and a mineral oil.

When the composition is formulated as a preparation, a diluent or excipient, such as a filler, an extender, a binder, a wetting agent, a disintegrant, or a surfactant, that are usually used may be used. Solid preparations for oral administration may include a tablet, a pill, a powder, granules, a capsule, and the like, and these solid preparations may be prepared by adding to the extract at least one excipient, for example, starch, calcium carbonate, sucrose or lactose, gelatin, or the like. In addition to simple excipients, lubricants such as magnesium stearate talc may also be used.

Preparations for oral administration correspond to a suspension, a liquid preparation, an emulsion, a syrup, and the like, and may comprise simple diluents that are frequently used, such as water and liquid paraffin, as well as several types of excipients, such as a humectant, a sweetener, an aroma, and a preservative.

Preparations for parenteral administration include a sterilized aqueous solution, a non-aqueous solvent, a suspension, an emulsion, a lyophilized agent, a suppository, a transdermal agent, and the like. Examples of the non-aqueous solvent and suspension may include propylene glycol, polyethylene glycol, a vegetable oil such as olive oil, an injectable ester such as ethylolate, and the like.

Examples of a substrate for the suppository may include Witepsol, Macrogol, Tween 61, cacao butter, laurin butter, glycerogelatin, and the like.

In an embodiment to which the composition of the present disclosure is applied to humans, the pharmaceutical composition of the present disclosure may be administered alone, but may be generally administered by mixing with a pharmaceutical carrier selected considering the manner of administration and standard pharmaceutical practice.

For example, the pharmaceutical composition of the present disclosure may be administered orally, intrabuccally, sublingually, in the form of a tablet comprising starch or lactose, in the form of a capsule with or without excipient; or in the form of an elixir or suspension comprising a chemical making a flavor or displaying a color.

These liquid preparations may be formulated with pharmaceutically acceptable additives, such as a suspension (for example, semi-synthesized glyceride such as methylcellulose, Witepsol, or a glyceride mixture such as a mixture of apricot kernel oil and PEG-6 ester or a mixture of PEG-8 and caprylic/capric glyceride).

The dose of the pharmaceutical composition of the present disclosure may vary depending on the age, body weight, sex of a patient, administration form, health condition, and severity of a disease, and may be divisionally administered at regular intervals according to the determination of the doctor and pharmacist.

For example, the daily dose, on the basis of the content of the active ingredient, may be 0.1 to 1000 mg/kg, 0.1 to 900 mg/kg, 0.1 to 800 mg/kg, 0.1 to 700 mg/kg, or 0.1 to 600 mg/kg, for example, 0.5 to 500 mg/kg. The dose is illustrative of the average case, and may be higher or lower depending on individual differences.

A daily dose of the pharmaceutical composition of the present disclosure, which is less than the above-described dose, cannot provide a significant effect, and a daily dose exceeding the above-described dose range is non-economical, and is out of the normal dose range and thus may cause an undesirable side effect, and therefore the above range is preferable.

### Advantageous Effects of Invention

The present disclosure relates to an M3 mutant peptide, a composition comprising the same as an active ingredient for alleviation, suppression, prevention, or treatment of a muscle disease, obesity, or diabetes, a method for alleviating, suppressing, preventing, or treating a muscle disease, obesity, or diabetes, by using an M3 mutant peptide, and use of an M3 mutant peptide for alleviating, suppressing, preventing, or treating a muscle disease, obesity, or diabetes.

### BRIEF DESCRIPTION OF DRAWINGS

FIG. 1A presents an image showing the results of examining the expression of mutant peptides through western blotting according to an exemplary embodiment of the present disclosure.

FIG. 1B presents a schematic diagram of mutant peptides according to an exemplary embodiment of the present disclosure.

FIG. 2 presents the results of examining the length of differentiation-induced myotubes of a control group through Jenner's staining according to an exemplary embodiment of the present disclosure.

FIG. 3 presents the results of examining the length of differentiation-induced myotubes of a wild type through Jenner's staining according to an exemplary embodiment of the present disclosure.

FIG. 4 presents the results of examining the length of differentiation-induced myotubes of an M3 mutant through Jenner's staining according to an exemplary embodiment of the present disclosure.

FIG. 5 presents a graph showing the results of examining the length of differentiation-induced myotubes through Jenner's staining according to an exemplary embodiment of the present disclosure.

FIG. 6 presents an image showing the results of examining the length of differentiation-induced myotubes of a control group through immunofluorescence staining according to an exemplary embodiment of the present disclosure.

FIG. 7 presents an image showing the results of examining the length of differentiation-induced myotubes of a wild type through immunofluorescence staining according to an exemplary embodiment of the present disclosure.

FIG. 8 presents an image showing the results of examining the length of differentiation-induced myotubes of an M3 mutant through immunofluorescence staining according to an exemplary embodiment of the present disclosure.

FIG. 9 presents an image showing the results of examining the length of differentiation-induced myotubes of an M4 mutant through immunofluorescence staining according to an exemplary embodiment of the present disclosure.

FIG. 10 presents an image showing the results of examining the length of differentiation-induced myotubes of an M5 mutant through immunofluorescence staining according to an exemplary embodiment of the present disclosure.

FIG. 11 presents an image showing the results of examining the length of differentiation-induced myotubes of an M6 mutant through immunofluorescence staining according to an exemplary embodiment of the present disclosure.

FIG. 12 presents a graph showing the results of examining the length of differentiation-induced myotubes through immunofluorescence staining according to an exemplary embodiment of the present disclosure.

FIG. 13 presents images showing the results of differentiation of adipose-derived mesenchymal stem cells into adipocytes according to an exemplary embodiment of the present disclosure.

FIG. 14 presents a graph showing the results of examining the extent of adipogenesis according to an exemplary embodiment of the present disclosure.

FIG. 15 presents a graph showing the results of measuring intracellular glucose uptake according to an exemplary embodiment of the present disclosure.

FIG. 16 presents a graph showing body weight changes in a muscle loss animal model according to an exemplary embodiment of the present disclosure.

FIG. 17 presents graphs showing the results of body weight changes of GA and TA muscle tissues in a muscle loss animal model according to an exemplary embodiment of the present disclosure.

FIG. 18 presents a graph showing fat and muscle weight changes in a muscle loss animal model according to an exemplary embodiment of the present disclosure.

FIG. 19 presents graphs showing the results of examining expression levels of MyoD, MyoG, Pax7, and MRF4 genes, which are muscle differentiation-related genes, according to an exemplary embodiment of the present disclosure.

FIG. 20 presents graphs showing the results of examining expression levels of MuRF1, Atrogin1, and Hes1, which are muscle differentiation inhibition-related genes, according to an exemplary embodiment of the present disclosure.

FIG. 21 presents a graph showing the results of examining the length of differentiation-induced myotubes through Jenner's staining according to an exemplary embodiment of the present disclosure.

FIG. 22 presents a graph showing the results of examining the extent of activity of myocytes according to an exemplary embodiment of the present disclosure.

### BEST MODE FOR CARRYING OUT THE INVENTION

Hereinafter, the present disclosure will be described in more detail with reference to exemplary embodiments. These exemplary embodiments are provided only for the purpose of illustrating the present disclosure in more detail, and therefore, according to the purpose of the present disclosure, it would be apparent to a person skilled in the art that these exemplary embodiments are not construed to limit the scope of the present disclosure.

### EXAMPLES

### Example 1: Culture and Differentiation Induction of C2C12 Cell Line

C2C12 cell line (ATCC, CRL-1772), a myoblast cell line mainly used for myocyte differentiation research, was cultured in DMEM supplemented with 10% fetal bovine serum (FBS), 100 U/ml penicillin, and 100 μg/ml streptomycin, as a culture medium, in a cell incubator at 37° C. supplied with 5% CO₂.

The C2C12 cell line was inoculated into 24-well cell culture plates at 5×10⁴/well and cultured in the culture medium. When the cells reached a density of 90% in the cell culture plates, the medium was replaced with DMEM comprising 2% horse serum as a differentiation medium and differentiation was induced. The differentiation medium was newly replaced at intervals of 3 days, and differentiation was induced in a cell incubator at 37° C. supplied with 5% CO₂ for a total of 8 days.

ADAMTS1 and mutant ADAMTS1 recombinant proteins produced in CHO-K1 cell line were subjected to isolate and purification by the method as in Example 2 below, and were used to treat the C2C12 cell line at concentrations of 0.01, 0.1, 1, 10, and 100 ng/ml once a day from the start of differentiation induction to the end of differentiation induction.

### Example 2: Production of Recombinant Protein

For isolation and purification of the recombinant proteins produced in the cells, ADAMTS1 overexpression vector was constructed so that 6×His was attached to the C-terminus of the recombinant proteins. Specifically, pEF6/V5-His A vector (Invitrogen, V96120) was digested with KpnI and XhoI restriction enzymes. The sequence recognized by KpnI was inserted at the 5′ end of the CDS part of the ADAMTS1 sequence (NM_006988.5) and the sequence recognized by XhoI was inserted at the 3′ end thereof, and then ligation to the previously digested vector was conducted using T4 DNA ligase. M1, M2, M3, M4, M5, and M6 mutant ADAMTS1 were constructed using wild-type ADAMTS1 as a template while starting from nucleotide number 664, nucleotide number 664-693 for M1, nucleotide number 664-723 for M2, nucleotide number 664-753 for M3, nucleotide number 664-783 for M4, nucleotide number 664-813 for M5, and nucleotide number 664-843 for M6 were cleaved, respectively, and in all the mutants, the sequences after nucleotide number 1237 were removed.

The CHO-K1 cell line was inoculated in 6-well cell culture plates using RPMI 1640 medium supplemented with 10% fetal bovine serum (FBS), 100 U/ml penicillin, 100 μg/ml streptomycin, and 25 mM HEPES as a culture medium and cultured in a cell incubator at 37° C. supplied with 5% CO₂. The next day, Lipofectamine 2000 (Invitrogen, 11668019) was mixed with the ADAMTS1 or ADAMTS1 mutant expression vectors and used to treat the CHO-K1 cells to introduce the vectors into the cells. After culturing for 24 hours, the cells were diluted 10-fold, transferred to 100 mm cell culture dishes, and then first treated with Blasticidin-S™ at a concentration of 10 μg/ml. Thereafter, the medium was replaced with a new culture medium mixed with Blasticidin-S™ at a concentration of 10 μg/ml a total of three times at intervals of 4 days. After independent colonies were formed by Blasticidin-S™, each colony was inoculated into new cell culture plates and cultured. Each colony was examined for whether ADAMTS1 or ADAMTS1 mutant proteins were expressed, through western blotting, and then used as a stabilized cell line. The CHO-K1 stabilized cell line was inoculated into 100 mm dishes, and when the cell density reached 100%, the medium was replaced with RPMI 1640 medium and cultured for 2 days. After 2 days, only the upper part of the culture was taken from the cultured dishes, purified with Ni-NTA agarose (Qiagen, 30210), recovered, and used for differentiation induction experiments of the C2C12 cell line. Specifically, the cell culture was passed through a gravity chromatography column (Qiagen, 34964) equipped with Ni-NTA agarose and then washed with 10 mM imidazole buffer. After repeated three times of washing, the proteins were purified by elution with 250 mM imidazole buffer.

### Example 3: Expression of Recombinant Proteins

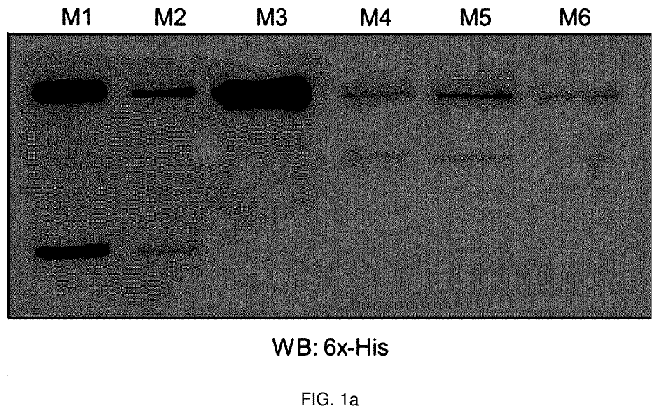

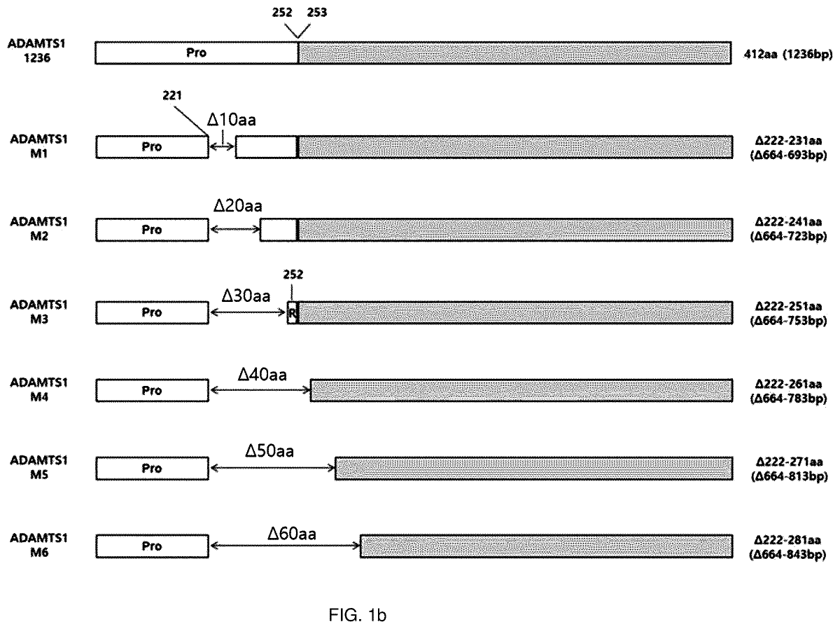

The culture of the ADAMTS1 mutant-overexpressed CHO-K1 stabilized cell line prepared by the method as in Example 2 was taken and examined for the expression of the constructed recombinant proteins through western blotting using an antibody recognizing 6×-His. The results are shown in FIGS. 1A and 1B.

As can be confirmed from FIG. 1B, M1 and M2 mutants showed that two types of proteins, a part comprising pro-domain and a mature domain, were all expressed, but M3, M4, M5, and M6 mutants showed that only a protein comprising a pro-domain was expressed. Out of the mutants expressing only the protein comprising a pro-domain, the others except for M3 mutant showed reduced expression (see FIG. 1A).

### Example 4: Jenner's Staining

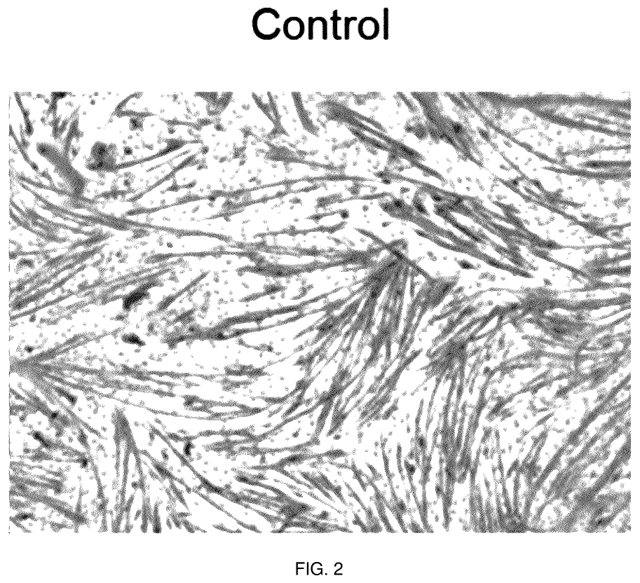

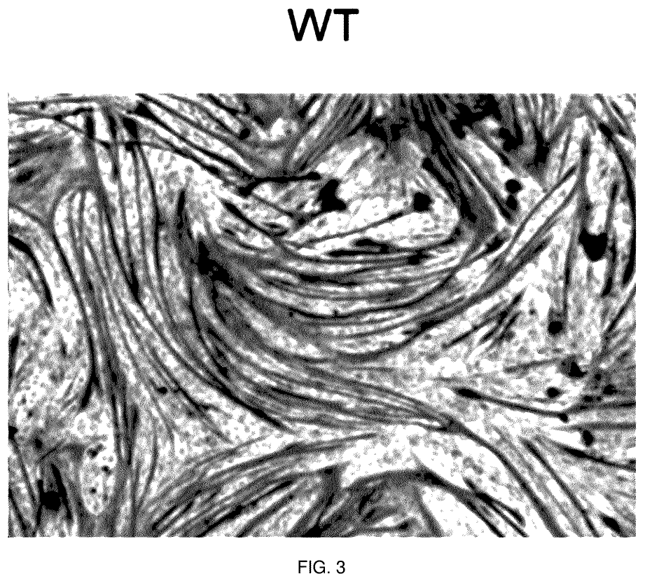

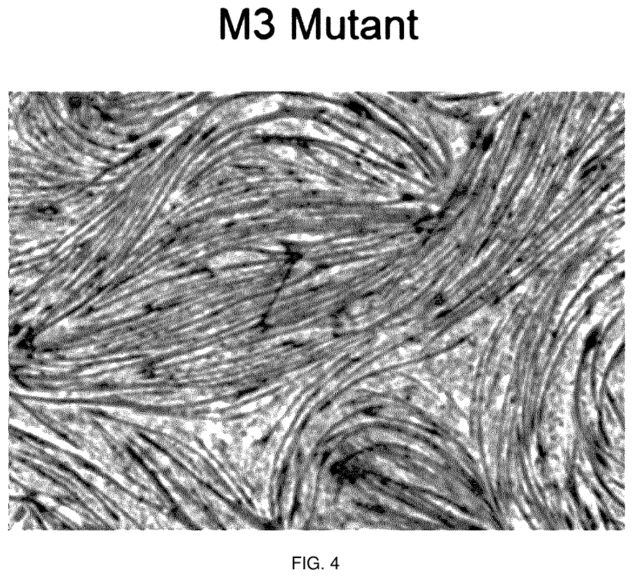

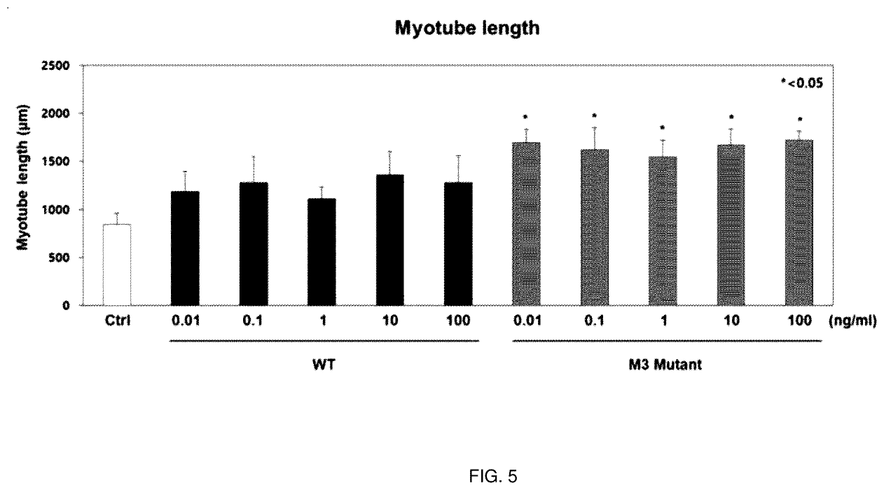

The C2C12 cell line was treated with ADAMTS1 or ADAMTS1 mutant recombinant protein while being differentiation-induced in 24-well culture plates for 8 days. The differentiation-induced cells were fixed in methanol for 10 minutes. The plates were sufficiently washed with PBS three times, and then stained for 10 minutes with a mixture of 2.5 mg/ml of Jenner staining reagent dissolved in methanol and distilled water at 1:1. The stained plates were washed with distilled water, followed by drying, and then observed under a microscope. The results are shown in FIGS. 2 to 5 and Table 1.

TABLE 1

| Classification | ng/ml | Mean (um) | SD |
|---|---|---|---|
| Ctrl | — | 840.5593 | 122.843 |
| WT | 0.01 | 1187.7 | 209.7841 |
| | 0.1 | 1279.783 | 270.5997 |
| | 1 | 1110.381 | 122.6237 |
| | 10 | 1361.083 | 238.143 |
| | 100 | 1276.93 | 279.3668 |
| M3 mutant | 0.01 | 1698.274 | 133.7239 |
| | 0.1 | 1618.237 | 230.9828 |
| | 1 | 1545.182 | 174.167 |
| | 10 | 1669.126 | 169.7136 |
| | 100 | 1718.463 | 94.99775 |

As can be confirmed from FIGS. 2 to 5 and Table 1, the co-treatment of C2C12 cell line with ADAMTS1 during the differentiation induction of the cells resulted in an increase in myotube length, and especially, the treatment with M3 mutant showed a more increase in myotube length compared with the differentiation-induced cell line treated with the wild type.

### Example 5: Immunofluorescence Staining

The C2C12 cell line treated with ADAMTS1 or ADAMTS1 mutant recombinant proteins while being differentiation-induced in 24-well culture plates for 8 days. The differentiation-induced cells were fixed in 4% formaldehyde for 10 minutes and then subjected to cell membrane permeabilization for 10 minutes by using 0.25% Triton X-100.

Thereafter, the plates were blocked with 2% BSA for 30 minutes, and then treated for 1 hour with the myosin heavy chain (MHC) antibody mixed with 2% BSA buffer at 1:500. Then, the cells were treated for 1 hour with Alexa Fluor® 555 fluorescence antibody mixed with 2% BSA at 1:200, and nuclei were stained through DAPI staining.

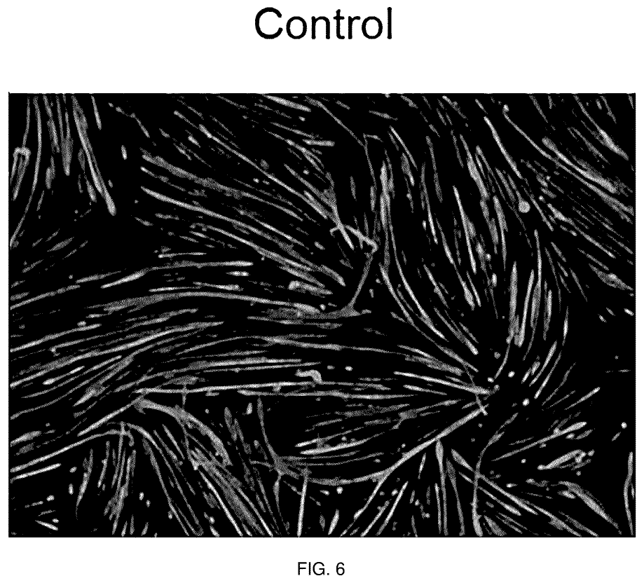

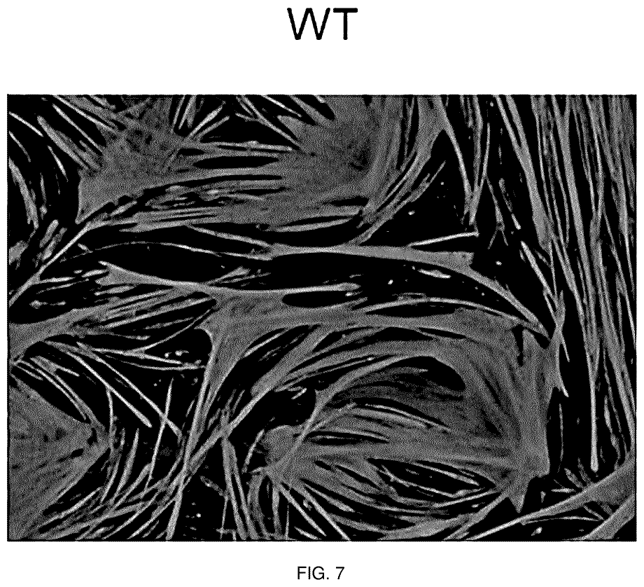

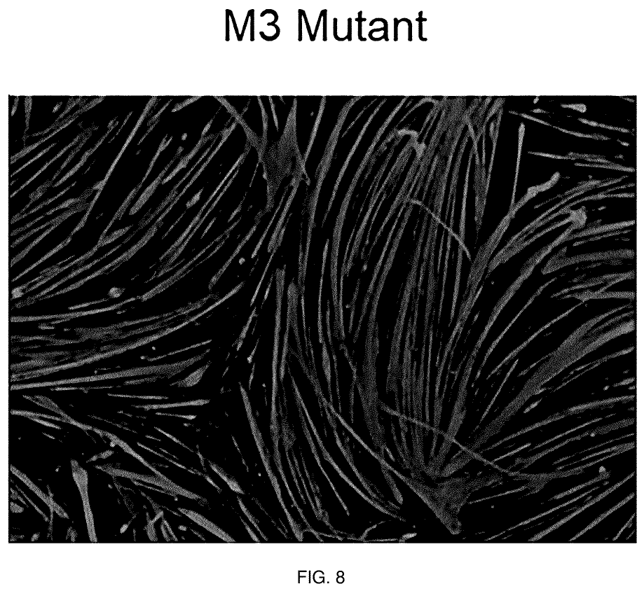

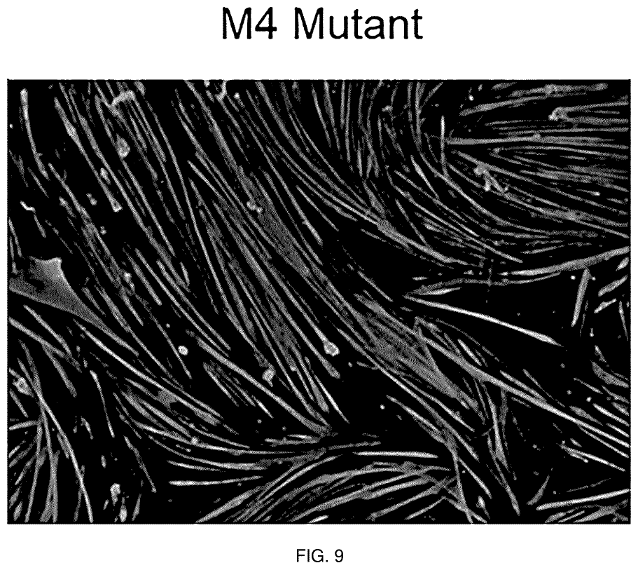

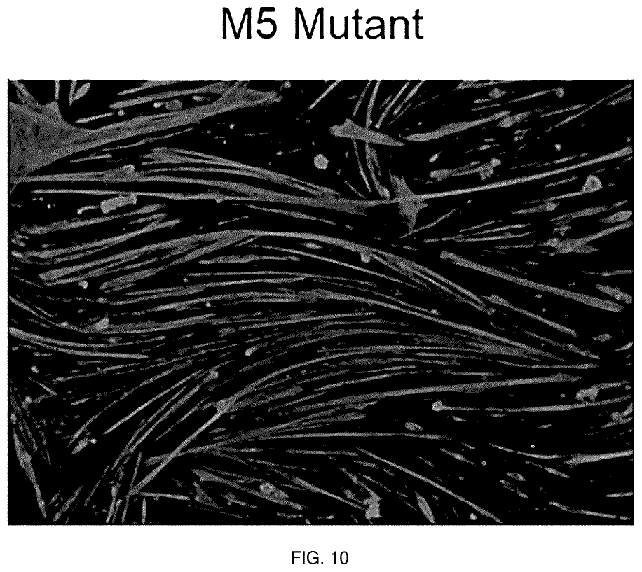

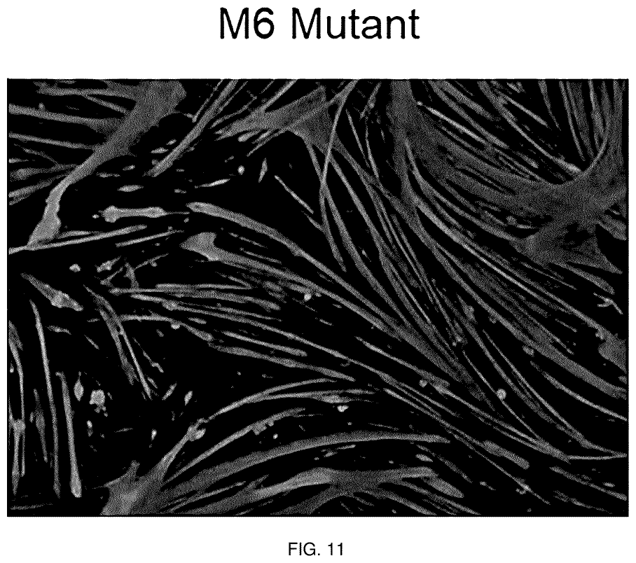

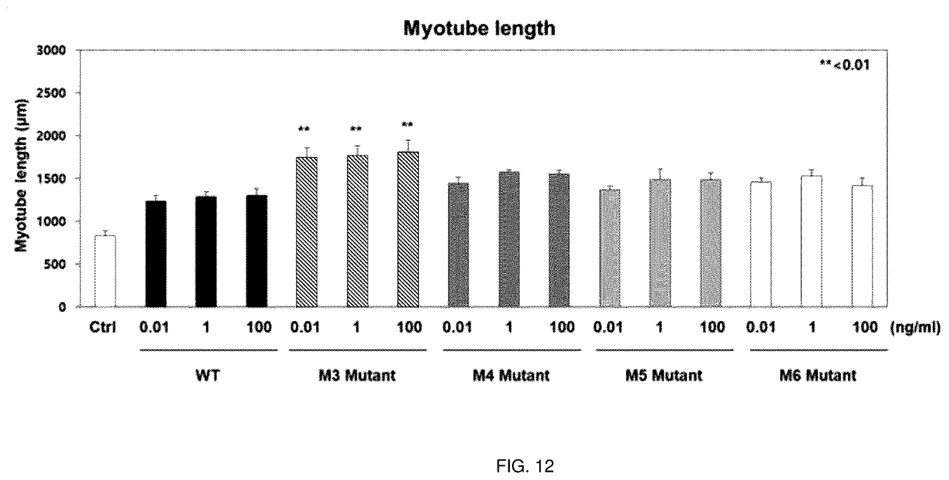

The stained plates were photographed using a fluorescence microscope (Nikon Ts2-FL), and the length of myotubes was measured using an analysis program (Nikon, NIS Elements) provided by the manufacturer and statistically processed. The analyzed plates were shielded from light and stored in a refrigerator. The results are shown in FIGS. 6 to 12 and Table 2.

TABLE 2

| Classification | ng/ml | Mean (μm) | Sd |
|---|---|---|---|
| Ctrl | — | 833.5567 | 58.78009 |
| WT | 0.01 | 1237.543 | 64.14704 |
| | 1 | 1286.943 | 59.92032 |
| | 100 | 1304.363 | 76.3394 |
| M3 mutant | 0.01 | 1739.657 | 122.5517 |
| | 1 | 1759.083 | 120.1073 |
| | 100 | 1809.76 | 141.1934 |
| M4 mutant | 0.01 | 1444.973 | 70.61137 |
| | 1 | 1573.837 | 27.24489 |
| | 100 | 1550.97 | 46.41372 |
| M5 mutant | 0.01 | 1365.46 | 48.78922 |
| | 1 | 1483.112 | 130.69 |
| | 100 | 1483.837 | 82.66543 |
| M6 mutant | 0.01 | 1455.1 | 52.42397 |
| | 1 | 1531.107 | 72.92847 |
| | 100 | 1418.417 | 88.17606 |

As can be confirmed from FIGS. 6 to 12 and Table 2, the differentiation-induced C2C12 cell lines treated with the ADAMTS1 wild type or mutants were differentiated into thicker and longer myotubes compared with the differentiation-induced C2C12 cell line treated with the differentiation medium.

In addition, the myotube length was longer in the cell lines treated with the ADAMTS1 mutants than the wild type, and especially, the longest myotubes were differentiation-induced by M3 mutant.

### Example 6: Extent of Adipogenesis

Adipose-derived mesenchymal stem cell line (ATCC, PCS-500-011) was cultured in a mesenchymal stem cell line exclusive medium (ATCC, PCS-500-030) comprising 2% fetal bovine serum, 5 ng/ml recombinant human epithelial cell factor, and 5 ng/ml recombinant human fibroblast growth factor, sold by ATCC. During cell differentiation, the medium was replaced with a medium in which StemPro medium (Gibco, A10410-01) was mixed with an additive (Gibco, A10065-01) provided by the manufacturer, and the differentiation into adipocytes was induced while the medium was replaced with new StemPro medium every 3 days for a total of 14 days. During the induction of differentiation, the cells were treated with ADAMTS1 wild type or M3 mutant recombinant protein at a concentration of 1 ng/ml or 100 ng/ml once daily.

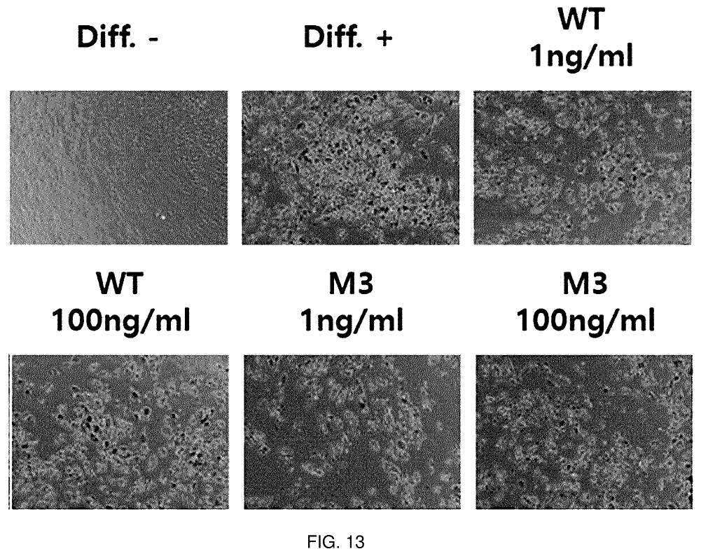

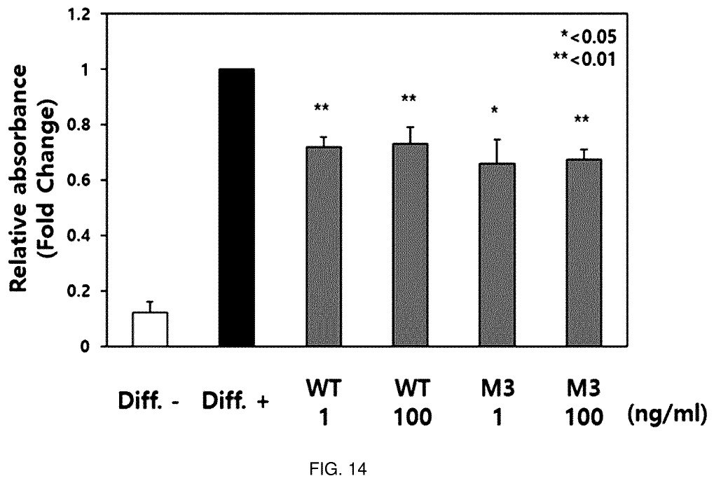

In order to examine the extent of adipogenesis from the differentiation-induced adipose-derived mesenchymal stem cells, Oil Red O staining was used. Specifically, the differentiated cells were fixed in 10% formalin and stained with Oil Red O staining solution. The stained cells were observed under a microscope, and then the deposited dye was extracted using 100% isopropanol. The extracted dye was quantified by measurement of absorbance at a wavelength of 520 nm, and the results are shown in FIGS. 13 and 14 and Table 3.

TABLE 3

| | | | WT 100 | | M3 100 |
|---|---|---|---|---|---|
| Diff. − | Diff. + | WT 1 (ng/ml) | (ng/ml) | M3 1 (ng/ml) | (ng/ml) |
| 0.120542 | 1 | 0.720773 | 0.731024 | 0.659033 | 0.67393 |
| 0.039364 | 0 | 0.032979 | 0.059734 | 0.086981 | 0.03776 |

As can be confirmed from FIGS. 13 and 14 and Table 3, both the wild type and M3 mutant of ADAMTS1 inhibited the differentiation of adipose-derived mesenchymal stem cells into adipocytes.

### Example 7: Intracellular Glucose Uptake

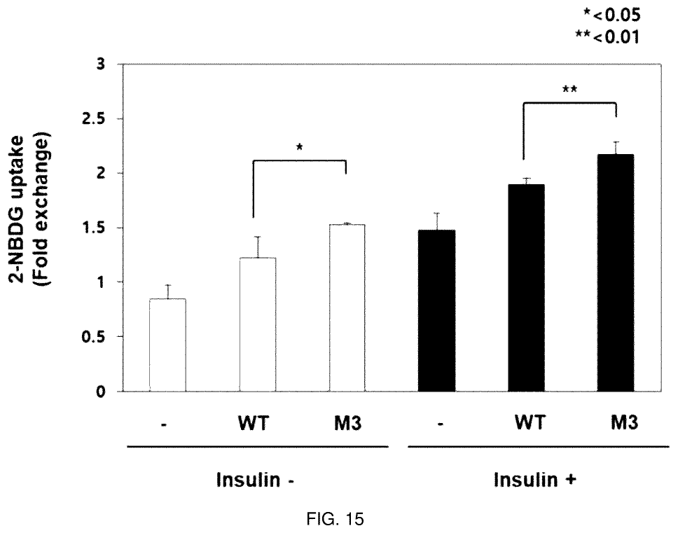

The intracellular glucose uptake that changes according to muscle differentiation increasing activity was measured using 2-(N-(7-nitrobenz-2-oxa-1,3-diazol-4-yl)amino-2-deoxyglucose (2-NBDG), a fluorescent glucose analogue. During the induction of differentiation for 8 days, C2C12 cell line was treated with ADAMTS1 or M3 mutant recombinant protein once daily at a concentration of 100 ng/ml, respectively. The differentiation-induced cells were treated with glucose-free DMEM for 2 hours, and thereafter, treated with 100 nM insulin for 15 minutes, and then 2-NBDG was added thereto to a final concentration of 100 μg/ml. The glucose uptake was measured at 485 nm for excitation and 535 nm for emission, and the results are shown in FIG. 15 and Table 4.

TABLE 4

| | Ins− | | | Ins+ | | |
|---|---|---|---|---|---|---|
| Classification | — | WT | M3 | — | WT | M3 |
| #1 | 1 | 1.297038 | 1.531155 | 1.246397 | 1.897571 | 2.269298 |
| #2 | 0.843478 | 1.31864 | 1.53383 | 1.508469 | 1.81309 | 2.228877 |
| #3 | 0.687519 | 1.33964 | 1.507814 | 1.60218 | 1.956826 | 2.177554 |
| #4 | 0.851029 | 0.92594 | 1.538645 | 1.544263 | 1.895672 | 2.009495 |
| Mean | 0.845506 | 1.220315 | 1.527861 | 1.475328 | 1.89079 | 2.171306 |
| SD | 0.127623 | 0.197019 | 0.013719 | 0.157428 | 0.05907 | 0.11422 |

As can be confirmed from FIG. 15 and Table 4, the extent of glucose uptake of the differentiation-induced C2C12 cell line increased under insulin treatment, and especially, the C2C12 cell line, for which the differentiation into myocytes was induced by the treatment with M3 mutant ADAMTS1, showed more increased glucose uptake compared with the cell line for which the differentiation was induced by the treatment with the wild type.

### Example 8: Weight Recovery in Muscle Loss Animal Model

12-Week-old C57BL/6 mice were administered with dexamethasone through intraperitoneal injection at a concentration of 50 mg/kg daily for 12 days. Simultaneously, the mice were administered through intraperitoneal injection with 100 μl of M3 mutant ADAMTS1 at concentrations of 0.02 μg/μl (Low), 0.2 μg/μl (Mid), and 2 μg/μl (High). The animal model was observed for weight recovery during administration for a total of 12 days.

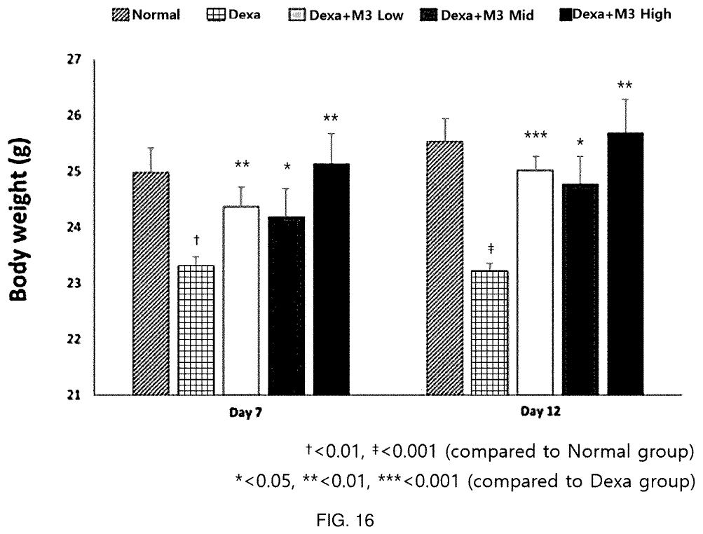

As can be confirmed from FIG. 16, the animal model with muscle loss induced by dexamethasone administration showed a distinctive weight loss compared with the normal group, and when the animal model was treated with M3 mutant ADAMTS1, all the Low, Mid, and High dose groups showed significant weight recovery. Especially, the weight recovery observed in the high dose group exceeded the normal level.

### Example 9: Weight Recovery of TA and GA Muscle Tissues in Muscle Loss Animal Model

To examine weight changes of muscle tissues in a muscle atrophy animal model obtained by administration of 12-week-old C57BL/6 mice with dexamethasone for 12 days, an autopsy was performed, after the completion of administration, to extract TA and GA muscle tissues. The extracted muscle tissues were weighed and compared.

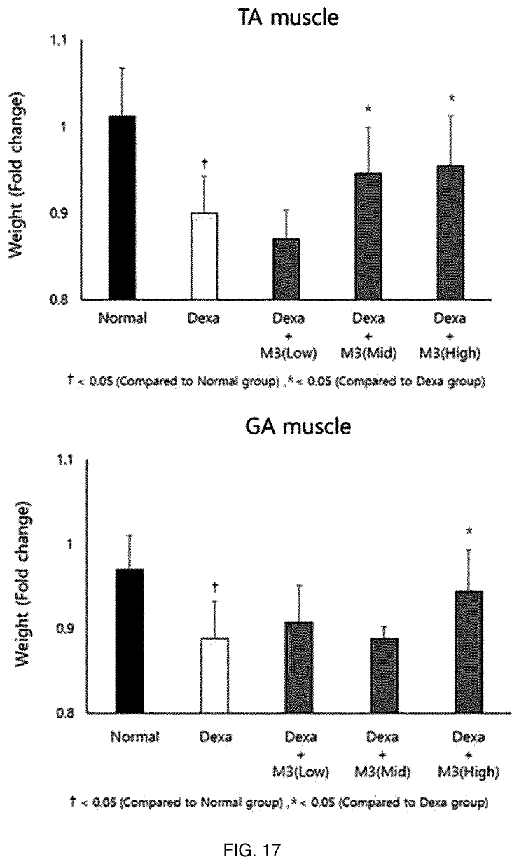

As can be confirmed from FIG. 17, the animal model with muscle atrophy induced by dexamethasone administration showed distinctive tissue weight losses of both of TA and GA tissues compared with the normal group, and when the muscle loss animal model was treated with M3 mutant ADAMTS1, the Mid dose group showed significant weight recovery of TA muscle tissue and the High dose group showed significant weight recovery of TA and GA muscle tissues.

### Example 10: Recovery of Fat and Muscle in Muscle Loss Animal Model

12-Week-old C57BL/6 mice were administered with dexamethasone through intraperitoneal injection at a concentration of 50 mg/kg daily for 12 days, and simultaneously, the mice were administered through intraperitoneal injection with 100 μl of M3 mutant ADAMTS1 at a concentration of 2 μg/μl. After the completion of administration, the mice were anesthetized through inhalational anesthesia and then analyzed for muscle mass and body fat mass by using a Dual-energy X-ray Absorptiometry (DXA) device.

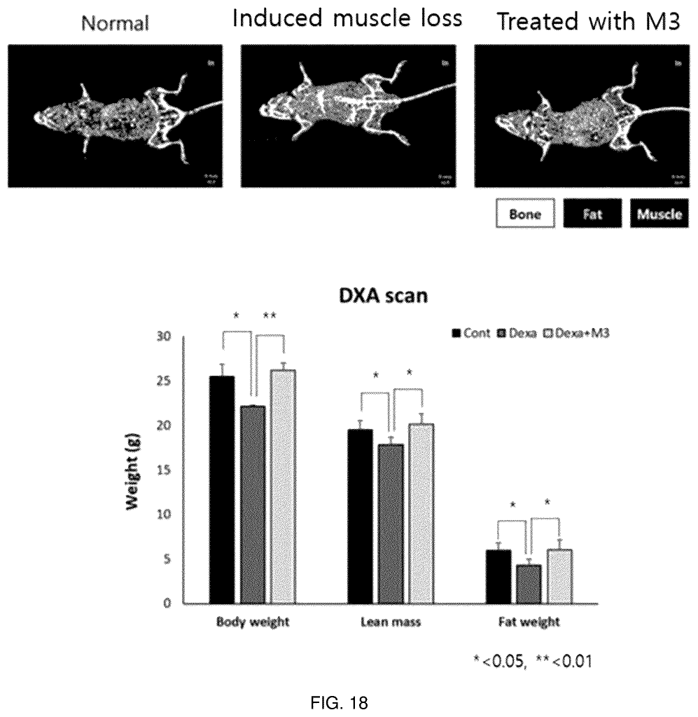

As can be confirmed from FIG. 18, both fat and muscle were significantly reduced in the animal model with muscle loss induced by dexamethasone administration. When the animal model with induced muscle atrophy was treated with M3 mutant ADAMTS1, both fat and muscle were recovered to the normal level.

### Example 11: Increases in Expression of Muscle Differentiation-Related Genes in Muscle Loss Animal Model

12-Week-old C57BL/6 mice were administered with dexamethasone through intraperitoneal injection at a concentration of 50 mg/kg daily for 12 days, and simultaneously, the mice were administered through intraperitoneal injection with 100 μl of M3 mutant ADAMTS1 at concentrations of 0.02 μg/μl (Low), 0.2 μg/μl (Mid), and 2 μg/μl (High). After the completion of administration of dexamethasone and M3 mutant ADAMTS1, an autopsy was performed to extract GA muscle tissue. The GA muscle tissue in a frozen state was homogenized using a pestle, and then RNA was extracted (Qiagen, 74104). The extracted RNA was prepared into cDNA using a cDNA synthesis reagent (Promega, A5000). The relative mRNA expression levels of MyoD, MyoG, Pax7, and MRF4, genes related to muscle differentiation, were compared from the synthesized cDNA by using a real-time polymerase chain reaction system (Applied Biosystems, Quantstudio3).

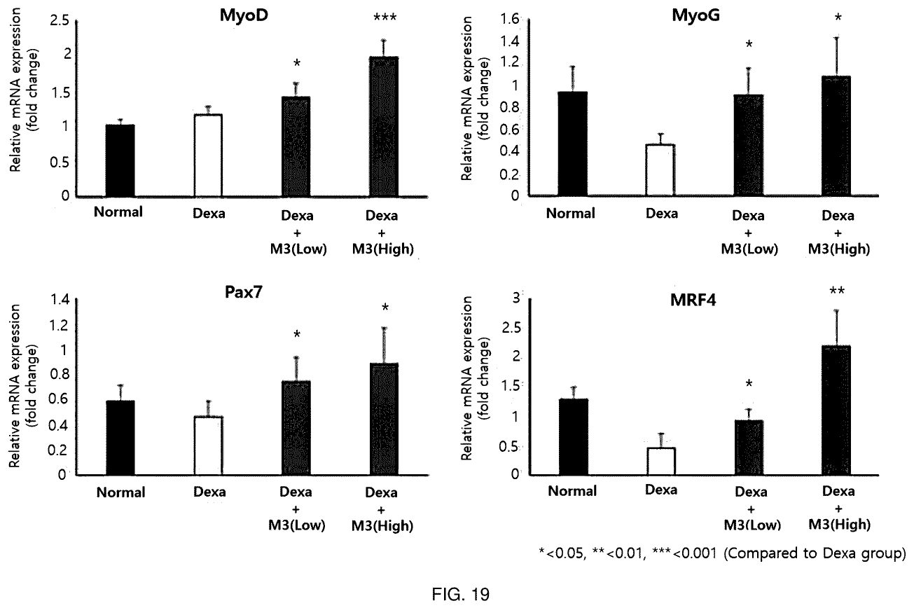

As can be confirmed from FIG. 19, when the animal model with muscle atrophy induced by dexamethasone treatment was treated with M3 mutant ADAMTS1, both the Low and High dose groups showed a significant increase in MyoD gene expression compared with the normal group and the dexamethasone treatment group.

Compared with the normal group, the dexamethasone treatment group showed significant decreases in MyoG, Pax7, and MRF4 expression levels, but the M3 mutant ADAMTS1 treatment showed significant increases in MyoG, Pax7, and MRF4 expression levels. This indicates that M3 mutant ADAMTS1 increased muscle differentiation by increasing the expression of muscle differentiation-related genes.

### Example 12: Reductions in Expression of Muscle Differentiation Inhibition-Related Genes in Muscle Loss Animal Model

12-Week-old C57BL/6 mice were administered with dexamethasone through intraperitoneal injection at a concentration of 50 mg/kg daily for 12 days, and simultaneously, the mice were administered through intraperitoneal injection with 100 μl of M3 mutant ADAMTS1 at concentrations of 0.02 μg/μl (Low), 0.2 μg/μl (Mid), and 2 μg/μl (High). After the completion of administration of dexamethasone and M3 mutant ADAMTS1, an autopsy was performed to extract GA muscle tissue. The GA muscle tissue in a frozen state was homogenized using a pestle, and then RNA was extracted (Qiagen, 74104). The extracted RNA was prepared into cDNA using a cDNA synthesis reagent (Promega, A5000). The relative mRNA expression levels of MuRF1, Atrogin1, and Hes1, which are muscle differentiation inhibition-related genes, were compared from the synthesized cDNA by using a real-time polymerase chain reaction system (Applied Biosystems, Quantstudio3).

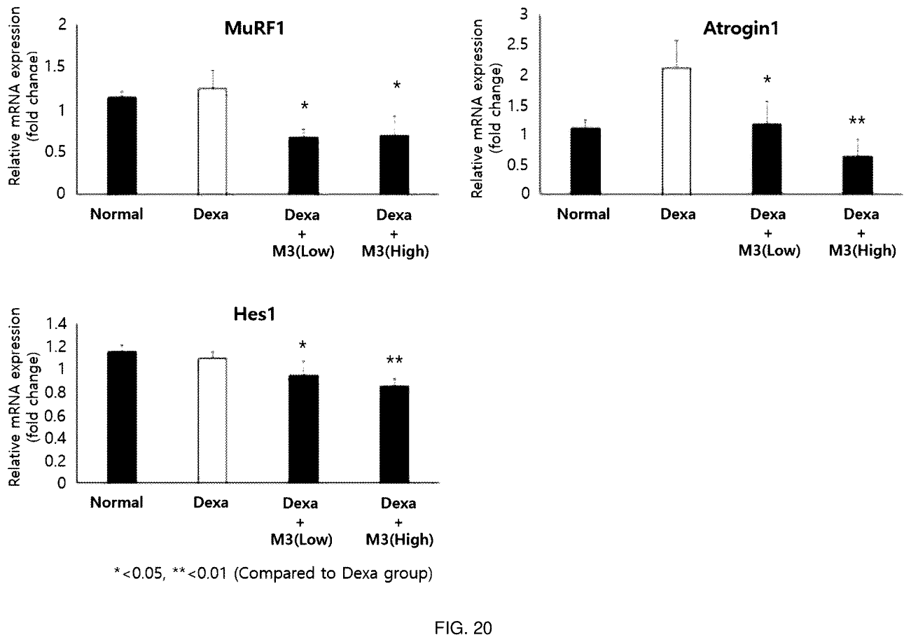

As can be confirmed from FIG. 20, when the animal model with muscle atrophy induced by dexamethasone treatment was treated with M3 mutant ADAMTS1, both the Low and High dose groups showed significant reductions in MuRF1, Atrogin1, and Hes1 gene expression. This indicates that M3 mutant ADAMTS1 can increase muscle differentiation by inhibiting muscle differentiation inhibition-related genes.

### Example 13: Increase in Muscle Differentiation in Cells with Muscle Differentiation Inhibition Induced by Anticancer Drug (Cancer Cachexia Model)

While the C2C12 myoblast cell line was differentiation-induced in 24-well cell culture plates for 8 days, the cells were treated with the cisplatin anticancer drug at a concentration of 50 μM together with M3 ADAMTS1 mutant recombinant protein at concentrations of 0.1, 1, and 10 ng/ml, respectively. The differentiation-induced cells were fixed in methanol for 10 minutes. The plates were sufficiently washed with PBS three times, and then stained for 10 minutes with a mixture of 2.5 mg/ml of Jenner staining reagent dissolved in methanol and distilled water at 1:1. The stained plates were washed with distilled water and then dried, followed by observation under a microscope.

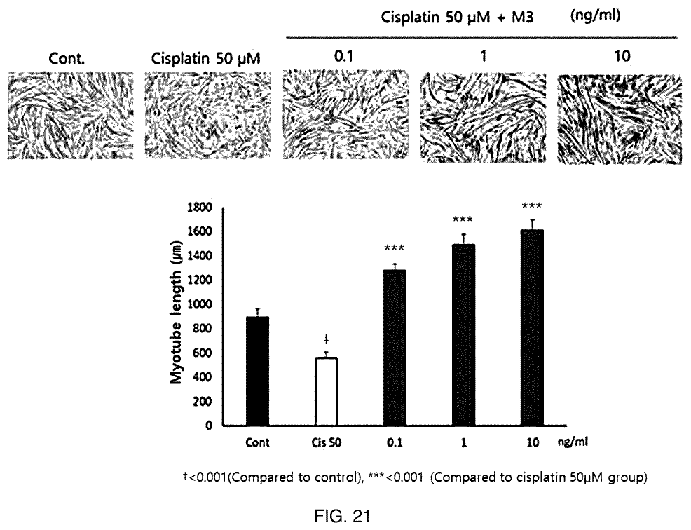

As can be confirmed from FIG. 21, when the cancer cachexia cell model with muscle differentiation inhibited by cisplatin treatment was treated with M3 mutant ADAMTS1, the shortened myotube length was significantly increased in a dose-dependent manner.

### Example 14: Increase in Activity of Primary Muscle Cells

Primary myocytes were cultured in 96-well plates for 24 hours, and then the medium was replaced with a serum-free medium, followed by further culturing for 24 hours. Thereafter, the cells were treated with 10 μM BrdU and M3 ADAMTS1 mutant protein at concentrations of 0.01, 0.1, 1, 10, and 100 ng/ml, followed by culturing for 24 hours. Then, the absorbance was measured at 450 nm by using a BrdU fluorescence measurement ELISA kit (Cell signaling technology, 6813S). An increase in the amount of newly synthesized DNA within the myocytes led to an increase in the measured absorbance, indicating an increase in activity of muscle cells.

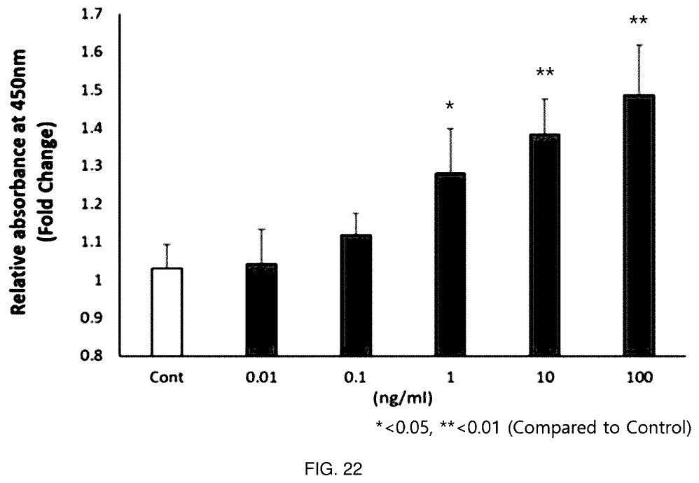

As can be confirmed from FIG. 22, the treatment with M3 mutant ADAMTS1 increased the activity of primary myocytes in a dose-dependent manner.

Although the present disclosure has been described in detail with reference to the specific features, it will be apparent to those skilled in the art that this description is only for a preferred embodiment and does not limit the scope of the present disclosure.

1. An M3 mutant peptide of a disintegrin and metalloprotease with thrombospondin motifs 1 (ADAMTS1), the peptide comprising a deletion of at least one amino acid selected from the group consisting of amino acids at positions 223 to 261 of a peptide consisting of the amino acid sequence of SEQ ID NO: 1.

2. The M3 mutant peptide of claim 1, wherein the M3 mutant peptide comprises a deletion of an amino acid at least one position selected from the group consisting of arginine at position 249, lysine at position 250, lysine at position 251, and arginine at position 252 of the peptide consisting of the amino acid sequence of SEQ ID NO: 1.

3. The M3 mutant peptide of claim 1, wherein the M3 mutant peptide comprises deletions of amino acids at positions 249 to 251 of the peptide consisting of the amino acid sequence of SEQ ID NO: 1.

4. The M3 mutant peptide of claim 1, wherein the M3 mutant peptide comprises i) a deletion of an amino acid at least one position selected from the group consisting of arginine at position 249, lysine at position 250, lysine at position 251, and arginine at position 252 and ii) a deletion of at least one amino acid selected from the group consisting of amino acids at positions 223 to 248 and 253 to 261, of the peptide consisting of the amino acid sequence of SEQ ID NO: 1.

5. The M3 mutant peptide of claim 1, wherein the M3 mutant peptide further comprises deletions of amino acids from aspartic acid at position 413 and serine at position 967 of the peptide consisting of the amino acid sequence of SEQ ID NO: 1.

6. The M3 mutant peptide of claim 1, wherein the M3 mutant peptide comprises deletions of amino acids at positions 223 to 251 and deletions of amino acids at positions 413 to 967 of the peptide consisting of the amino acid sequence of SEQ ID NO: 1.

7. The M3 mutant peptide of claim 6, wherein the M3 mutant peptide consists of the amino acid sequence of SEQ ID NO: 2.

8.-11. (canceled)

12. A method for treating a muscle disease, obesity, or diabetes, comprising:
administering to a subject in need thereof a composition comprising the peptide of claim 1.

13. The method of claim 12, wherein the muscle disease is selected from the group consisting of sarcopenia, amyotrophy, cancer cachexia, muscle injury, muscular dystrophy, atrophy of heart, atony, muscular dystrophy, muscular degeneration, and myasthenia.


SEQUENCE LISTING (4 sequences)

SEQ ID NO: 1 - protein, 967 aa, Artificial Sequence
  ADAMTS1
MQRAVPEGFGRRKLGSDMGNAERAPGSRSFGPVPTLLLLAAALLAVSDALGRPSEEDEELVVPELERAPGHGTTRLRLHAFDQQLDLELRPDSSFLAPGFTLQNVGRKSGSETPLPETDLAHCFYSGTVNGDPSSAAALSLCEGVRGAFYLLGEAYFIQPLPAASERLATAAPGEKPPAPLQFHLLRRNRQGDVGGTCGVVDDEPRPTGKAETEDEDEGTEGEDEGAQWSPQDPALQGVGQPTGTGSIRKKRFVSSHRYVETMLVADQSMAEFHGSGLKHYLLTLFSVAARLYKHPSIRNSVSLVVVKILVIHDEQKGPEVTSNAALTLRNFCNWQKQHNPPSDRDAEHYDTAILFTRQDLCGSQTCDTLGMADVGTVCDPSRSCSVIEDDGLQAAFTTAHELGHVFNMPHDDAKQCASLNGVNQDSHMMASMLSNLDHSQPWSPCSAYMITSFLDNGHGECLMDKPQNPIQLPGDLPGTSYDANRQCQFTFGEDSKHCPDAASTCSTLWCTGTSGGVLVCQTKHFPWADGTSCGEGKWCINGKCVNKTDRKHFDTPFHGSWGMWGPWGDCSRTCGGGVQYTMRECDNPVPKNGGKYCEGKRVRYRSCNLEDCPDNNGKTFREEQCEAHNEFSKASFGSGPAVEWIPKYAGVSPKDRCKLICQAKGIGYFFVLQPKVVDGTPCSPDSTSVCVQGQCVKAGCDRIIDSKKKFDKCGVCGGNGSTCKKISGSVTSAKPGYHDIITIPTGATNIEVKQRNQRGSRNNGSFLAIKAADGTYILNGDYTLSTLEQDIMYKGVVLRYSGSSAALERIRSFSPLKEPLTIQVLTVGNALRPKIKYTYFVKKKKESFNAIPTFSAWVIEEWGECSKSCELGWQRRLVECRDINGQPASECAKEVKPASTRPCADHPCPQWQLGEWSSCSKTCGKGYKKRSLKCLSHDGGVLSHESCDPLKKPKHFIDFCTMAECS

SEQ ID NO: 2 - protein, 382 aa, Artificial Sequence
  M3
MQRAVPEGFGRRKLGSDMGNAERAPGSRSFGPVPTLLLLAAALLAVSDALGRPSEEDEELVVPELERAPGHGTTRLRLHAFDQQLDLELRPDSSFLAPGFTLQNVGRKSGSETPLPETDLAHCFYSGTVNGDPSSAAALSLCEGVRGAFYLLGEAYFIQPLPAASERLATAAPGEKPPAPLQFHLLRRNRQGDVGGTCGVVDDEPRPTGKAETEDEDEGTERFVSSHRYVETMLVADQSMAEFHGSGLKHYLLTLFSVAARLYKHPSIRNSVSLVVVKILVIHDEQKGPEVTSNAALTLRNFCNWQKQHNPPSDRDAEHYDTAILFTRQDLCGSQTCDTLGMADVGTVCDPSRSCSVIEDDGLQAAFTTAHELGHVFNMPHD

SEQ ID NO: 3 - DNA, 2904 bp, Artificial Sequence
  ADAMTS1
atgcagcgagctgtgcccgaggggttcggaaggcgcaagctgggcagcgacatggggaacgcggagcgggctccggggtctcggagctttgggccagtacccacgctgctgctgctcgccgcggcgctactggccgtgtcggacgcactcgggcgcccctccgaggaggacgaggagctagtggtgccggagctggagcgcgccccgggacacgggaccacgcgcctccgcctgcacgcctttgaccagcagctggatctggagctgcggcccgacagcagctttttggcgcccggcttcacgctccagaacgtggggcgcaaatccgggtccgagacgccgcttccggaaaccgacctggcgcactgcttctactccggcaccgtgaatggcgatcccagctcggctgccgccctcagcctctgcgagggcgtgcgcggcgccttctacctgctgggggaggcgtatttcatccagccgctgcccgccgccagcgagcgcctcgccaccgccgccccaggggagaagccgccggcaccactacagttccacctcctgcggcggaatcggcagggcgacgtcggcggcacgtgcggggtcgtggacgacgagccccggccgactgggaaagcggagaccgaagacgaggacgaagggactgagggcgaggacgaaggggctcagtggtcgccgcaggacccggcactgcaaggcgtaggacagcccacaggaactggaagcataagaaagaagcgatttgtgtccagtcaccgctatgtggaaaccatgcttgtggcagaccagtcgatggcagaattccacggcagtggtctaaagcattaccttctcacgttgttttcggtggcagccagattgtacaaacaccccagcattcgtaattcagttagcctggtggtggtgaagatcttggtcatccacgatgaacagaaggggccggaagtgacctccaatgctgccctcactctgcggaacttttgcaactggcagaagcagcacaacccacccagtgaccgggatgcagagcactatgacacagcaattcttttcaccagacaggacttgtgtgggtcccagacatgtgatactcttgggatggctgatgttggaactgtgtgtgatccgagcagaagctgctccgtcatagaagatgatggtttacaagctgccttcaccacagcccatgaattaggccacgtgtttaacatgccacatgatgatgcaaagcagtgtgccagccttaatggtgtgaaccaggattcccacatgatggcgtcaatgctttccaacctggaccacagccagccttggtctccttgcagtgcctacatgattacatcatttctggataatggtcatggggaatgtttgatggacaagcctcagaatcccatacagctcccaggcgatctccctggcacctcgtacgatgccaaccggcagtgccagtttacatttggggaggactccaaacactgccccgatgcagccagcacatgtagcaccttgtggtgtaccggcacctctggtggggtgctggtgtgtcaaaccaaacacttcccgtgggcggatggcaccagctgtggagaagggaaatggtgtatcaacggcaagtgtgtgaacaaaaccgacagaaagcattttgatacgccttttcatggaagctggggaatgtgggggccttggggagactgttcgagaacgtgcggtggaggagtccagtacacgatgagggaatgtgacaacccagtcccaaagaatggagggaagtactgtgaaggcaaacgagtgcgctacagatcctgtaaccttgaggactgtccagacaataatggaaaaacctttagagaggaacaatgtgaagcacacaacgagttttcaaaagcttcctttgggagtgggcctgcggtggaatggattcccaagtacgctggcgtctcaccaaaggacaggtgcaagctcatctgccaagccaaaggcattggctacttcttcgttttgcagcccaaggttgtagatggtactccatgtagcccagattccacctctgtctgtgtgcaaggacagtgtgtaaaagctggttgtgatcgcatcatagactccaaaaagaagtttgataaatgtggtgtttgcgggggaaatggatctacttgtaaaaaaatatcaggatcagttactagtgcaaaacctggatatcatgatatcatcacaattccaactggagccaccaacatcgaagtgaaacagcggaaccagaggggatccaggaacaatggcagctttcttgccatcaaagctgctgatggcacatatattcttaatggtgactacactttgtccaccttagagcaagacattatgtacaaaggtgttgtcttgaggtacagcggctcctctgcggcattggaaagaattcgcagctttagccctctcaaagagcccttgaccatccaggttcttactgtgggcaatgcccttcgacctaaaattaaatacacctacttcgtaaagaagaagaaggaatctttcaatgctatccccactttttcagcatgggtcattgaagagtggggcgaatgttctaagtcatgtgaattgggttggcagagaagactggtagaatgccgagacattaatggacagcctgcttccgagtgtgcaaaggaagtgaagccagccagcaccagaccttgtgcagaccatccctgcccccagtggcagctgggggagtggtcatcatgttctaagacctgtgggaagggttacaaaaaaagaagcttgaagtgtctgtcccatgatggaggggtgttatctcatgagagctgtgatcctttaaagaaacctaaacatttcatagacttttgcacaatggcagaatgcagttaa

SEQ ID NO: 4 - DNA, 1146 bp, Artificial Sequence
  M3
atgcagcgagctgtgcccgaggggttcggaaggcgcaagctgggcagcgacatggggaacgcggagcgggctccggggtctcggagctttgggccagtacccacgctgctgctgctcgccgcggcgctactggccgtgtcggacgcactcgggcgcccctccgaggaggacgaggagctagtggtgccggagctggagcgcgccccgggacacgggaccacgcgcctccgcctgcacgcctttgaccagcagctggatctggagctgcggcccgacagcagctttttggcgcccggcttcacgctccagaacgtggggcgcaaatccgggtccgagacgccgcttccggaaaccgacctggcgcactgcttctactccggcaccgtgaatggcgatcccagctcggctgccgccctcagcctctgcgagggcgtgcgcggcgccttctacctgctgggggaggcgtatttcatccagccgctgcccgccgccagcgagcgcctcgccaccgccgccccaggggagaagccgccggcaccactacagttccacctcctgcggcggaatcggcagggcgacgtcggcggcacgtgcggggtcgtggacgacgagccccggccgactgggaaagcggagaccgaagacgaggacgaagggactgagcgatttgtgtccagtcaccgctatgtggaaaccatgcttgtggcagaccagtcgatggcagaattccacggcagtggtctaaagcattaccttctcacgttgttttcggtggcagccagattgtacaaacaccccagcattcgtaattcagttagcctggtggtggtgaagatcttggtcatccacgatgaacagaaggggccggaagtgacctccaatgctgccctcactctgcggaacttttgcaactggcagaagcagcacaacccacccagtgaccgggatgcagagcactatgacacagcaattcttttcaccagacaggacttgtgtgggtcccagacatgtgatactcttgggatggctgatgttggaactgtgtgtgatccgagcagaagctgctccgtcatagaagatgatggtttacaagctgccttcaccacagcccatgaattaggccacgtgtttaacatgccacatgat